(19) United States
(12) Patent Application Publication
(10) Pub. No.: US 2008/0279958 A1
(43) Pub. Date: Nov. 13, 2008

(54) SNAKE VENOM COMPOSITIONS AND METHODS OF USE

(76) Inventors: William H. Reeves, Coral Springs, FL (US); Ruben P. Laguens, La Plata (AR); William J. Marsheck, Margate, FL (US); Martin Laguens, La Plata (AR)

Correspondence Address: AKERMAN SENTERFITT, P.O. BOX 3188, WEST PALM BEACH, FL, 33402-3188, US

(21) Appl. No.: 12/118,030

(22) Filed: May 9, 2008

Related U.S. Application Data
(60) Provisional application No. 60/916,923, filed on May 9, 2007.

(51) Int. Cl. A61K 35/58; A61P 35/00; A61P 19/02; A61P 37/00

(52) U.S. Cl. 424/542

(57) ABSTRACT

The invention relates to the discovery that formulations of Lachesis venom decrease circulating TNF-α levels in mammals including humans, and that administration of Lachesis venom-containing formulations to mammals suffering from sepsis, parasitic infection, cisplatin nephrotoxicity, rheumatoid arthritis, cancer and AIDS mitigates symptoms associated with these conditions.

### CROSS-REFERENCE TO RELATED APPLICATIONS

The present application claims the priority of U.S. provisional patent application No. 60/916,923 filed on May 9, 2007.

### FIELD OF THE INVENTION

The invention relates generally to the fields of medicine, molecular biology and herpetology. More particularly, the invention relates to compositions and methods for modulating circulating Tumor Necrosis Factor (TNF-α) levels in an animal subject suffering from a disease or condition in which circulating TNF-α levels play a role.

### BACKGROUND

TNF-α is a proinflammatory cytokine produced primarily by macrophages but also by a broad variety of mammalian cell-types including lymphoid cells, mast cells, endothelial cells, fibroblasts, and neuronal tissue. A number of disease conditions exist in which elevated circulating levels of TNF-α play a role in the pathology of the disease. Examples of such diseases include cancer, immunodeficiency syndromes, asthma, septicemia, endotoxic shock, rheumatoid and septic arthritis, infectious diseases, hepatitis, chronic diarrhea, psoriasis, Crohn's disease, ulcerative colitis, chronic intoxications, viral and severe alcoholic hepatitis, and intestinal parasites.

An effective treatment capable of modulating TNF-α levels in patients suffering from diseases and conditions in which TNF-α plays a significant role and mitigating the symptoms associated with such diseases and conditions is highly desirable.

### SUMMARY

The invention relates to the discovery that formulations of Lachesis venom decrease circulating TNF-α levels in mammals including humans, and that administration of Lachesis venom-containing formulations or formulations containing one or more component(s) of Lachesis venom to mammals suffering from sepsis, parasitic infection, cisplatin nephrotoxicity, rheumatoid arthritis, cancer and Acquired Immune Deficiency Syndrome (AIDS) mitigates symptoms associated with these conditions. Furthermore, the data described below gathered from human subjects suggest treatment of other similarly diseased mammals with Lachesis venom or component(s) thereof would be beneficial (e.g., Feline Immunodeficient Virus (FIV) in cats, rheumatoid arthritis in dogs and horses, etc.).

Lachesis is the venom of the snake Lachesis muta muta (common names include surucucu and bushmaster) whose natural habitat is the rain forests of Central and South America. The venom is composed of several enzymes (e.g., Lachesis Hemorrhagic factor, gyroxin, protease 1, several disintegrins, and phospholipase A2) which affect clotting factors, fibrinolytic proteins, and the plasmatic kinin system. Compositions as described herein typically include Lachesis venom diluted in water and/or alcoholic solutions (e.g., 0.5-2% aqueous alcohol solutions) at a concentration of about 30 to about 320 (e.g., 25, 30, 40, 50, 60, 70, 80, 90, 100, 120, 150, 170, 200, 220, 250, 270, 300, 320) picogram/kilogram (pg/km) depending on the disease or condition to be treated and can be administered via any suitable route. In one embodiment, Lachesis venom diluted in an aqueous-alcoholic medium exhibited protective effects on different tissues in animal subjects by mitigating or preventing cellular damage caused by an excess of circulating TNF-α, either by reducing TNF-α production or blocking TNF-α action. The Lachesis venom-containing compositions described herein can be used to treat animal subjects suffering from a wide variety of diseases and conditions in which circulating TNF-α level plays a role.

Accordingly, the invention features a method for modulating circulating TNF-α levels in an animal subject having a disease or condition in which circulating TNF-α levels are elevated. The method includes the step of administering a composition including Lachesis venom or at least one component thereof and a pharmaceutically acceptable carrier and/or excipient to the animal subject in an amount sufficient to modulate (e.g., decrease) circulating TNF-α levels in the animal subject. The animal subject can have one or more of the following conditions: cancer, Chagas' disease, rheumatoid arthritis (RA), cisplatin nephrotoxicity, sepsis, and an immunodeficiency disease (AIDS).

Unless otherwise defined, all technical terms used herein have the same meaning as commonly understood by one of ordinary skill in the art to which this invention belongs.

The phrase “modulating circulating TNF-α levels in an animal subject” means eliciting an increase or decrease in the amount of circulating TNF-α levels and/or increasing/reducing the activity of TNF-α (e.g., by binding to TNF-α or receptors of TNF-α in an animal subject). An increase or decrease in circulating TNF-α levels in an animal subject can be measured by TNF-α expression and/or TNF-α activity.

When referring to Lachesis venom or at least one component thereof, the term “purified” refers to a component of Lachesis venom that is substantially separated from other components in the venom. For example, a protein or plurality of proteins naturally occurring in Lachesis venom that modulate TNF-α can be purified from a sample of Lachesis venom. A purified sample of Lachesis venom typically has a higher specific activity than a non-purified sample. Lachesis venom components can be purified by any suitable means, including size-exclusion chromatography, ion-exchange chromatography, and gel-electrophoresis chromatography.

As used herein the phrase “elevated circulating TNF-α levels” means levels of circulating TNF-α in a mammalian subject having a disease or condition in which elevated circulating TNF-α levels play a role (e.g., cause the disease or condition or are a symptom or side effect of the disease) that are increased (e.g., higher than normal physiological levels) compared to circulating TNF-α levels in a mammalian subject not having the condition or disease. The level of circulating TNF-α in a mammalian subject having a disease or condition in which elevated circulating TNF-α levels play a role depends on the type of mammal, the disease or condition, as well as other factors such as age and weight of the mammal.

Although methods and compositions similar or equivalent to those described herein can be used in the practice or testing of the present invention, suitable methods and compositions are described below. All publications, patent applications, patents, and other references mentioned herein are incorporated by reference in their entirety. In the case of conflict, the present specification, including definitions, will control. The particular embodiments discussed below are illustrative only and not intended to be limiting.

### DETAILED DESCRIPTION

The invention provides compositions and methods for modulating circulating TNF-α levels in an animal subject suffering from a disease or condition in which circulating TNF-α levels play a role. Compositions for modulating circulating TNF-α levels as described herein include Lachesis venom and a pharmaceutically acceptable carrier and/or excipient. Among the uses which have been found for the compositions and methods described herein are chronic enteropathies, malabsorption syndrome caused by enteric parasites, acute hepatitis of diverse origin, chemical intoxication of diverse origin, antidote for diverse intoxications, treatment of septicemia, treatment of AIDS, cancer, treatment of diverse infectious diseases, psoriasis and skin-related disorders, psoriatic arthritis, auto-immune arthritis, immune arthritis, treatment of vascular injuries in shock cases, as an adjuvant along with conventional therapies for the above-mentioned uses, as an adjuvant in the process of post-operative recovery of major surgeries, as a prophylaxis in the immediate post-operative phase of major surgeries, as an adjuvant in the treatment of burn patients, as a chemopreventive of TNF-α-mediated tissue damage in anti-neoplastic drug usage, and in all conditions in which TNF-α plays a significant role in the pathogenetic mechanisms of tissue injury.

The below-described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 2003 (with periodic updates).

### Disease and Conditions in Which Elevated Circulating TNF-α Levels Play A Role

Described herein are methods of modulating circulating TNF-α levels in an animal subject having a disease or condition in which elevated circulating TNF-α levels play a role. The methods described herein can be used to treat an animal subject having any disease or condition in which elevated circulating TNF-α causes one or more symptoms of the disease or condition or exacerbates the disease or condition. A non-exhaustive list of diseases and conditions in which elevated circulating TNF-α levels play a role follows.

In the acute inflammatory response to gram-negative bacteria and other infectious microbes, TNF-α is the principal mediator. In severe infections, TNF-α is produced in large amounts and causes systemic clinical and pathological abnormalities. For example, sepsis is a complex disease characterized by an increased inflammatory response in the body's attempt to combat an infection from microorganisms such as bacteria, fungi, or viruses. A weak host inflammatory response can lead to greater infection, whereas an excessive inflammatory response may lead to tissue damage, myocardial injury, acute respiratory failure, multiple organ failure or death. Controlling inflammation is, thus, a central focus of treating sepsis. The incidence of sepsis in the United States ranges from 400,000 to 750,000 cases per year. Mortality due to sepsis is approximately 30 percent and increases with age from 10 percent in children to 40 percent in the elderly. Mortality is 50 percent or greater in patients with the more severe syndrome, septic shock. Septic shock is due to the production of TNF-α and other cytokines, including IL-12, IFN-γ, and IL-1. Today, septic shock is the most frequent cause of death in intensive care units, with about 100,000 deaths per year in the United States. Most of the cases are due to gram-negative endotoxin-producing bacteria, such as Escherichia coli, Klebsiella pneumonia, Proteus species, Pseudomonas auroginosa and serratia, Bacteroides, etc., but gram-positive bacteria, such as Pneumoccus, Streptococcus, and Staphylococcus may elicit a similar response. Additionally, some fungi can produce a similar syndrome. Antagonists of TNF-α have been shown to prevent mortality in animal models, but clinical trials with anti-TNF-α antibodies or with soluble TNF-α receptors have not shown benefits in patients with sepsis.

Another condition in which TNF-α plays a role is arthritis. Arthritis and other rheumatic conditions (AORC) are the leading cause of disability in the United States. The cost of AORC in the United States is $116.3 billion annually (i.e., $51.1 billion in direct costs plus $65.2 billion in indirect costs), approximately 1.4% of the U.S. gross domestic product. One of the pathogenetic mechanisms of damage in rheumatoid arthritis is related to an increase in systemic and intraarticular TNF-α levels. No effective treatment exists.

TNF-α is thought to play a role in Chagas' heart disease is unclear. Chagas's heart disease is caused by the protozoan Typanosoma Cruzi, and is a common cause of cardiomyopathy in the Americas. It has been reported that during the initial stages of infestation, TNF-α plays a protective effect by increasing macrophagic trypanomicide activity by means of an increased synthesis of nitric oxide (Santos Lima et al., Infect Immun 1997; 65: 457-465). At later stages of acute infestation, however, an increase in TNF-α synthesis induces cachexia and a deleterious effect among animals (Truyens et al., Parasite Immunol, 1995; 17: 561-568; Truyens et al., Infect Immun, 1999; 67: 5579-5586). A majority of patients with Chagas' disease remain in the asymptomatic latent phase of disease for 10 to 30 years or even for life (70% of patients). Mortality during the acute phase of Chagas' disease is around 5 percent. Five-year mortality of chronic Chagas' disease with cardiac dysfunction is above 50 percent. In acute phase, death is mostly caused by myocarditis, and in chronic phase it is mostly by irreversible cardiomyopathy. Specific anti-Chagas' therapy with trypanocide drugs is useful in the acute phase, but the trypanocidal treatment management of chronic Chagas' heart disease at present is not universally accepted.

Cisplatin nephrotoxicity is thought to be mediated by increased TNF-α production. Cisplatin is one of the major drugs used in antineoplastic chemotherapy. Cisplatin is a chemotherapy agent used alone or in combination with other agents to treat metastatic testicular or ovarian cancer, Hodgkin's disease, non-Hodgkin's lymphoma, brain tumors, cancer of the nervous system and cancer of the head, neck, bone, cervical, lung and bladder cancer. Unfortunately, nephrotoxicity and cumulative renal insufficiency are dose-limiting factors of cisplatin chemotherapy.

It is known that many clinical manifestations, e.g. cachexia, of terminal cancer are due to an increase in circulating TNF-α. Prolonged production of TNF-α causes cachexia, a wasting that occurs from TNF-α-induced appetite suppression and reduced synthesis of lipoprotein lipase, an enzyme needed to release fatty acids from circulating liposomes so that they can be used by tissues. Cachexia can often accompany advanced heart failure. Elevated circulating levels of TNF-α has been shown in these cases.

It is also known that different concomitant infections in HIV-positive patients produce an activation of macrophages which in turn produce more TNF-α, rendering an increase in viral titers because TNF-α favors the transcription of HIV messenger RNA.

Finally, TNF-α is a cytokine involved in the regulation of body carbohydrate and lipid metabolism. It may induce an increase in serum cholesterol and hepatic 3-methyl-glutaryl coenzyme reductase. The levels of TNF-α have been compared in obese and normal weight human subjects.

### Compositions for Modulating Circulating TNF-α Levels

Compositions for modulating circulating TNF-α levels in an animal subject include Lachesis venom or a component thereof and a pharmaceutically acceptable carrier and excipient(s). Lachesis venom is commercially available as a lyophilized powder and can be used in any suitable formulation (e.g., lyophilized powder, aqueous solution, gel, cream, etc.). In a typical composition for modulating circulating TNF-α levels in an animal subject, a measured quantity of powdered Lachesis venom active ingredient is diluted in phosphate buffered saline (PBS) at a concentration of about 0.01 μg/ml. The concentration of Lachesis venom in a composition for modulating circulating TNF-α levels in an animal subject is dependent upon the disease or condition to be treated. For example, in a typical composition for treating an animal subject having sepsis, septic shock, RA, cisplatin nephrotoxicity, cancer, AIDS, or Chagas' heart disease, the concentration of Lachesis venom is about 30 pg/kg (e.g., 30 pg/kg diluted in an appropriate amount of buffer or saline such as PBS). For purposes of delivery by injection, it is preferable that a PBS dilution be used. PBS maintains pH in a range close to a physiological one and is also iso-osmotic when compared with plasma. This means that the number of salts diluted in PBS is similar to that of body fluid, so no harmful effect will be expected because of the vehicle. In the examples described below, Lachesis venom-containing compositions included dilutions ranging from 10⁻² to 10⁻¹⁰ of the extract in water and/or aqueous solutions (e.g. PBS), and in the range of about 0.5-2% in alcoholic solutions. The compositions described herein can be administered to an animal subject one or more times a day, depending on the type and severity of disease or condition to be treated. Any suitable pharmaceutically acceptable carrier can be used in compositions and methods as described herein. Pharmaceutically acceptable carriers are described in greater detail below.

A Lachesis venom-containing composition can be prepared by a number of methods. In a typical method, Lachesis venom is commercially obtained, diluted in an appropriate buffer, sterilized (e.g., by filtration through a 0.22 micron membrane), and stored at −20° C. until use. Any suitable buffer can be used (e.g., PBS, citrate buffer, bicarbonate buffer, etc.) and any suitable sterilization process can be used (e.g., gamma-radiation, pasteurization, ultraviolet light, filtration, etc.). In some embodiments, one or more preservatives (e.g., sodium or potassium sorbate, sodium benzoate, citrate, etc.), anti-oxidants, additives, and stabilizers can be added to a Lachesis venom-containing composition. Lachesis venom-containing compositions can be prepared as galenicals. Lachesis venom-containing compositions can be prepared just prior to use, or can be prepared for long-term storage prior to use.

Lachesis venom-containing compositions may decrease circulating TNF-α levels in an animal by decreasing TNF-α production (e.g., gene expression) or by decreasing existing TNF-α levels (e.g., by degrading or promoting the degradation of TNF-α).

### Methods of Modulating Circulating TNF-α Levels In a Subject

Described herein are methods of modulating circulating TNF-α levels in an animal subject having a disease or condition in which elevated circulating TNF-α levels play a role. A typical method includes the step of administering a composition including Lachesis venom or at least one component thereof and a pharmaceutically acceptable carrier and/or a pharmaceutically acceptable excipient to an animal subject having a disease or condition in which elevated circulating TNF-α levels play a role (e.g., exacerbate) in the disease or condition. The composition is administered to the animal subject in an amount sufficient to decrease circulating TNF-α levels in the subject. In some embodiments, Lachesis venom or the at least one component thereof is an adjuvant and is administered to an animal subject with another therapeutic agent as a combination therapy.

### Subjects

An animal subject as described herein is any animal into which a composition for modulating circulating TNF-α levels can be administered. In general, animals such as mammals (e.g., human beings, dogs, cats, pigs, sheep, mice, rats, rabbits, cattle, goats, horses, etc.) are suitable subjects. The terms subject and animal subject are used herein interchangeably.

### Lachesis Venom-Containing Compositions Used as Adjuvants

Lachesis venom and Lachesis venom-containing compositions and formulations as described herein can also be used as adjuvants for other pharmaceutical products or therapeutic methods, increasing the effectiveness of the pharmaceutical products or therapeutic methods, reducing dosage of the pharmaceutical products, and/or reducing the time of administration/application of the specific pharmaceutical products or therapeutic methods (radiotherapy for instance, among others).

### Effective Doses

The compositions described above are preferably administered to a mammal in an effective amount, that is, an amount capable of producing a desirable result in a treated subject (e.g., modulating circulating TNF-α levels in the subject). Such a therapeutically effective amount can be determined as described below.

Toxicity and therapeutic efficacy of the compositions described herein can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration.

As is well known in the medical arts, dosage and mode of administration for any one subject depends on many factors, including the subject's condition or disease state, size, body surface area, age, the particular composition to be administered, time and route of administration, general health, and other drugs being administered concurrently.

### Administration of Compositions

Compositions as described herein can be administered to a mammal (e.g., humans) by any suitable route, e.g., parenteral, enteric, topical, intrathecal, transcelomic, intrarticular, sublingual, transdermal, intranasal, inhalation, and subcutaneous. For example, when treating enteric pathologies, compositions as described herein can be administered either orally or rectally. As another example, skin lesions can be treated by topical administration (solutions, creams, etc.), while systemic conditions can be treated by parenteral administration (subcutaneous, intramuscular, intraperitoneal or intravenous injection). In a typical method of administration, a Lachesis venom-containing composition is administered to a mammal by injection.

To facilitate delivery of compositions to a mammal, a composition can be mixed with a carrier or excipient. Carriers and excipients that might be used include saline (especially sterilized, pyrogen-free saline), saline buffers (e.g., citrate buffer, phosphate buffer, acetate buffer, and bicarbonate buffer), amino acids, urea, alcohols, ascorbic acid, phospholipids, proteins (e.g., serum albumin), EDTA, sodium chloride, liposomes, mannitol, lactose, sorbitol, dextrins, and glycerol. Lachesis venom formulations or venom component(s) compositions may be inserted into an osmotic pump device or attached to polymeric material for slow release into the body as in cases of subcutaneous or transdermal delivery systems. USP grade carriers and excipients are particularly useful for administration of Lachesis venom-containing compositions as described herein. Methods for making such formulations are well known and can be found in, for example, Remington's Pharmaceutical Sciences, 18^{th} ed., by Alfonso R. Gennaro, Mack Pub. Co. (Easton, Pa.), 1995.

### EXAMPLES

The present invention is further illustrated by the following specific examples. The examples are provided for illustration only and should not be construed as limiting the scope of the invention in any way.

### Example 1

### Treating Sepsis Shock In Mammals

Bacterial LPS-induced endotoxic shock is a well-established model to evaluate sepsis pathogenetic pathways and different treatment schedules.

### Material and Methods

Inbred 8 week-old male Balb-c mice were used. Bacterial lipopolysaccharides from Salmonella enteridis and Lachesis venom were purchased from Sigma Co. (St. Louis, Mo.).

### Experimental Schedule

The mice were divided in 4 groups of 6 animals each. Three groups received 0.5 ml of a solution containing LPS (400 ug/animal) by intra-peritoneal route. The remaining groups received 0.5 ml of PBS by the same route. Group A received 0.5 ml of PBS containing 30 pg/kg of Lachesis venom (e.g., catalogue no. V7376 Sigma-Aldrich, St. Louis, Mo.) by intra-peritoneal route 30 minutes after LPS inoculation. Group B received 0.5 ml of PBS containing 300 pg/kg of Lachesis venom in a similar schedule. Group C received only 0.5 ml of PBS (i.p.) 30 minutes after LPS inoculation. Group D was used as a control group, receiving only Lachesis (300 pg/kg). Morbimortality was recorded every 8 hours for 4 consecutive days. Eighteen hours after LPS inoculation, blood samples were obtained, and plasmatic TNF-α levels were determined by means of an ELISA* commercial kit (PharMingen, San Diego, Calif.). Dead animals were submitted to a necropsy procedure if no signs of stiffness were present. Surviving animals were sacrificed at day 4 and a necropsy was performed. Samples of different organs were obtained for histological study.

### Results

Five animals out of 6 that received LPS and PBS (Group C) died between 18 and 48 hours after the beginning of the experiment, while 3 animals out of 6 of each group that received LPS and 30 pg/kg or 300 pg/kg of Lachesis venom (groups A and B, respectively) died between 36 and 60 hours after LPS inoculation.

Four hours after inoculation, Group C animals showed acute illness signs characterized by lethargy, fluffy hair, and diarrhea. Groups A and B showed a better physical status, and characteristic signs appeared at 12 hours after LPS inoculation with a quick improvement of the clinical condition in those surviving animals. Group D animals showed no signs of disease at all and no mortality was recorded. All spontaneously dead animals (regardless of which Group they belonged to) showed, on histological observation, signs of disseminated intravascular coagulation with perivascular bleeding in different tissues, accumulation of polymorphonuclear neutrophil leucocytes in lung alveolar walls and hepatic sinuses, and renal tubular necrosis. In those surviving animals killed 4 days after LPS inoculation, no morphological alterations were recorded.

Plasma TNF-α values are summarized in the following Table 1:

| | Group A | Group B | Group C | Group D* |
|---|---|---|---|---|
| | 1424 | 1992 | 2581 | <23.4 |
| | 1531 | 1548 | 3201 | <23.4 |
| | 1584 | 1442 | 2314 | 30.9 |
| | 1958 | 1495 | 3008 | <23.4 |
| | 1869 | 1548 | 2492 | <23.4 |
| | 1768 | 1948 | 2719 | <23.4 |
| Mean | 1689** | 1662.16** | 2719.2** | N.D. |

*TNF-α values were under the detection threshold of the kit employed for the determinations.
**p: <0.000251369 for Group A compared with Control group C. p: <0.00029344 for Group B compared with Control group C.
TNF-α values are expressed as Umol/l.

### Conclusions

Lachesis administration in the two concentrations used significantly inhibited endogenous TNF-α production. These lower levels of TNF-α were associated with lack of morphological damage and an increased survival. Lachesis protected against TNF-α-mediated damage induced by LPS. No significant differences could be detected between 30 and 300 pg/kg doses.

### Example 2

### Effect of Lachesis Venom on Mouse Experimental Acute Chagas' Disease

In order to evaluate the activity of Lachesis venom on a murine model of acute infection with the protozoan Trypanosoma Cruzi, the causative agent of South American Trypanosomiasis (Chagas' heart disease), 8 week-old female mice received 2500 trypomastigots of T. cruzi Tulahuen strain by intraperitoneal route.

Two groups of nine animals received 0.2 ml of PBS containing 30 pg/kg of Lachesis venom by subcutaneous route every other day for 3 or 7 weeks. Infested control animals received PBS by subcutaneous route. Morbimortality, number of circulating parasites, plasmatic levels of anti-T.cruzi antibodies, and intensity of myocardium and skeletal muscle damage as well as spleen morphology were recorded. One group of experimental and control animals were sacrificed at day 21 post-infestation while the other groups were used for mortality recording.

### Results

By week 5, control animals showed a marked weight loss, with hair loss on head and scattered over the body. Lachesis-treated animals showed mild weight loss with lesser hair loss. Mortality in the control group was high: six out of nine animals died, while mortality among Lachesis-treated animals was minimal, with one out of nine animals dying (p=0.0002).

At 21 days post-infestation, the number of circulating parasites was significantly lesser in-Lachesis treated animals than in control ones (p=0.0001).

At 21 days post-infestation, myocarditis and myositis were of minor intensity in Lachesis-treated animals as compared with control ones (p=0.02 and p=0.05, respectively). Results are shown in Table 2.

At 21 days post-infestation, plasmatic levels of anti-T cruzi antibodies were significantly higher in Lachesis-treated animals than control ones (p=0.02). Results are shown in Table 3.

Differences in plasma cell counts were recorded in this experiment. Lachesis-treated animals presented more plasma cell in splenic white pulp than control animals (p=0.05). Results are shown in Table 4.

### Conclusions

Low-dose Lachesis administration significantly lowered circulating parasite number, tissue lesions, and mortality of acute infested mice with a pathogenetic strain of T. cruzi. This effect appears to be mediated by an increase in the humoral immune response against T. cruzi as shown by the increase in specific titers of antibodies and increase in splenic plasma cells. It is possible that Lachesis exerts some action on the cytokine cascade that is operating in various steps of the immune response. The results presented herein show that Lachesis treatment reduces TNF-α secretion in mouse peritoneal macrophages.

TABLE 2
Cardiac and muscular lesions.

| | Heart | | Skeletal muscle | |
|---|---|---|---|---|
| | Control | Lachesis | Control | Lachesis |
| | 3 | 1 | 4 | 3 |
| | 2 | 1 | 3 | 3 |
| | 1 | 0 | 3 | 3 |
| | 0 | 2 | 2 | 2 |
| | 4 | 2 | 3 | 2 |
| | 3 | 1 | 3 | 1 |
| | 2 | 3 | 2 | 1 |
| | 3 | 0 | 3 | 0 |
| | 4 | 1 | 4 | 2 |
| Mean | 2.44 | 1.35* | 3 | 1.8** |
| SEM | 0.14 | 0.1 | 0.07 | 0.2 |

Each piece of data corresponds to one animal.
*p < 0.02
**p < 0.05, compared with respective controls.
Score used: 0: no lesion. 1: Slight 2: Mild; 3: Moderate; 4: Severe

TABLE 3
Plasmatic titers of anti-T.cruzi antibodies
at day 21 post-infestation.

| | Control | Lachesis |
|---|---|---|
| | 64 | 128 |
| | 128 | 256 |
| | 32 | 512 |
| | 256 | 512 |
| | 64 | 1024 |
| | 64 | 256 |
| | 128 | 256 |
| | 32 | 64 |
| | 32 | 128 |
| Mean | 88.8 | 348.4* |
| SEM | 8.1 | 33.1 |

Antibody titers are expressed as the inverse of the last positive dilution.
*p < 0.02, as compared with controls.

TABLE 4
Number of splenic plasma cells per mm² of
tissue as observed under a microscope.

| | Control | Lachesis |
|---|---|---|
| | 86 | 143 |
| | 54 | 112 |
| | 67 | 98 |
| | 90 | 165 |
| | 44 | 68 |
| | 59 | 134 |
| | 71 | 116 |
| | 33 | 93 |
| | 56 | 77 |
| Mean | 62.22 | 111.77 |
| SEM | 1.93 | 3.44* |

*p < 0.05, as compared with controls.

### Example 3

### Effect of Lachesis Venom on Systemic Plasma Cell in a Mouse Model

In order to determine the number and distribution of plasma cells in the spleen of mice treated with highly diluted Lachesis venom, 8 inbred male 8 week-old Balb-c mice received subcutaneously 0.2 ml of PBS containing 30 pg/kg of Lachesis every other day, for 21 days. As a control, 8 mice received PBS on a similar schedule. Simultaneously, with the first administration of Lachesis, experimental and control animals were challenged with bacterial protein antigens of commercial origin. At day 23 all animals were sacrificed, and the spleen removed. The spleen was weighed and then processed with routine histological techniques.

### Results

Although spleen weight was similar between experimental and control groups, histological appearance of white pulp was clearly different. Non-treated animals presented a follicular hyperplasia, with well-developed germinal centers and prominent macrophages containing basophyllic material in their cytoplasms. A clearly demarcated border was noted between the germinal center and the surrounding lymphocytic crown which, in turn, was hyperplastic. Lachesis treated animals showed also a hyperplastic white pulp, but border between germinal center and lymphocytic crown was not easily evident. Germinal centers were not so prominent. An increase was noted in the number of plasma cells, more evident in periarteriolar sleeves.

Immunophenotyping for plasma cells was performed, showing a significant increase in the number of plasmocytes in treated animals with respect to control ones. Studies have shown that TNF-α-deficient mice present structural changes in their white pulp. It is reasonable to speculate that Lachesis would modify TNF-α values in immunostimulated animals, leading this decay to structural changes in white pulp with an increase in plasma cells. (Cook et al., J Exp Med 1998; 188: 1503-1511).

### Example 4

### Effects of Highly Diluted Lachesis Venom on Renal Toxicity of Cisplatin

Since Lachesis venom presents a reducing effect on TNF-α production in experimental endotoxic shock, its possible protective effect on cisplatin nephrotoxicity was explored.

### Experimental Design

Inbred 8 week-old male Balb-c mice received 20 or 50 mg/kg of cisplatin by intraperitoneal route. Twenty-four hours before, half of the animals were divided into 2 groups, and each group received an intraperitoneal injection of PBS containing 30 or 300 pg/kg of Lachesis venom. As controls, the remaining animals received PBS in a similar schedule. Sixty hours after cisplatin administration, animals were sacrificed and samples of blood for urea and creatinine plasma concentration determinations were obtained. Samples of lymph nodes, kidney, liver, gut, lung, bone marrow, and spleen for histological examination were also obtained.

### Results

Lachesis administration significantly reduced tubular damage in the kidney as well as neutrophil leukocyte infiltration. These lesions are characteristic of cisplatin toxicity. Other organs showed no lesions. In the following tables it can be observed that Lachesis administration, at a concentration of 30 pg/kg, significantly reduced urea and creatinine plasma levels, thus indicating a protective effect against this drug toxicity, rendering a tendency to normalization of functional altered parameters. It appears that the more diluted the Lachesis, the better the effect. This protective effect was observed in both doses of cisplatin used.

TABLE 5
Creatinine and urea serum values in Lachesis or PBS treated
mice that received 20 mg/kg of body weigh of cisplatin.
Note the striking differences between lachesis and placebo
(PBS) treated groups (P value for creatinine: 0.005804880,
and P value for urea: 0.00011162).

| | LACHESIS GROUP | | PBS GROUP | |
|---|---|---|---|---|
| Animal | creatinine | urea | creatinine | urea |
| 1 | 2.02 | 2.64 | 1.91 | 5.22 |
| 2 | 1.01 | 2.64 | 2.41 | 5.38 |
| 3 | 0.69 | 2.40 | 2.87 | 5.31 |
| 4 | 0.60 | 3.11 | 2.87 | 5.31 |
| 5 | 0.74 | 3.06 | 3.14 | 4.99 |
| 6 | 0.15 | 0.59 | 3.32 | 5.94 |
| 7 | 0.11 | 0.39 | 0.66 | 4.21 |
| 8 | 0.28 | 2.51 | 0.82 | 3.50 |
| 9 | 0.53 | 3.31 | 0.53 | 3.04 |
| 10 | 0.30 | 1.97 | 0.3 | 1.62 |
| Mean | 0.643 | 2.262 | 1.77333333 | 4.35666667 |
| SD | 0.561111 | 1.0116651 | 1.21200248 | 1.39587786 |
| SEM | 0.16 | 0.3 | 0.4 | 0.46 |

TABLE 6
Creatinine and urea serum values of mice that receive 50 mg/kg of body weight
of Cisplatin. Animals were divided in 3 groups, one group was treated with
300 pg/g of Lachesis by intraperitoneal route, other group received 30 pg/kg
of Lachesis by the same way and the last group received PBS and served as
a control. Although both Lachesis treated groups showed a decay in creatinine
and urea values as compared with control group, those animals receiving the
minor dose showed the more striking differences.

| | LACHESIS 300 pg/g | | LACHESIS 30 pg/g | | PBS | |
|---|---|---|---|---|---|---|
| ANIMAL | creatinine | urea | creatinine | urea | creatinine | urea |
| 1 | 0.76 | 2.07 | 0.61 | 1.47 | 3.10 | 5.37 |
| 2 | 0.73 | 3.04 | 0.75 | 1.85 | 2.89 | 5.14 |
| 3 | 2.09 | 4.01 | 0.70 | 2.28 | 0.68 | 2.74 |
| 4 | 0.90 | 3.02 | | | 2.55 | 5.01 |
| MEAN | 1.12 | 3.0575 | 0.6866666 | 1.8666666 | 2.305 | 4.560000 |

### Example 5

### Modulating TNF-α Production In Macrophages

In order to evaluate Lachesis venom as a potential treatment for arthritis and other rheumatic conditions, TNF-α production in cultured murine macrophages treated with Lachesis venom was analyzed in the following experiment.

Peritoneal macrophages were obtained from 2-3 month old male inbred Balb-c mice by means of peritoneal washings. Cells obtained in this manner were placed in plastic Petri dishes and allowed to attach to the plastic surface for 2 hours.

After rinsing, 200 ul of a solution containing 1 mg×10⁻⁷/ml of Lachesis venom was added to each culture. After one hour of incubation, the cultures were rinsed twice to remove the Lachesis solution. Supernatant was collected at 1, 2, 3, 6, 12, 18, and 24 hours post-treatment. ELISA assays were performed on each sample to measure TNF-α production. As a control, alternative cultures were treated with a aqueous-alcoholic solution without Lachesis.

### Results

Almost 80 percent of the treated macrophages showed morphological changes typical of activated cells, while only 20 percent of the control cells showed this affect. Activated cells were defined as those showing expanded cytoplasmic prolongations with large and vesiculated nuclei while quiescent cells were defined as those of rounded shape with scarce prolongations and small, condensed nuclei.

TNF-α could be detected in these cultures only at 18 and 24 hours post-treatment. Previous times showed no TNF-α production, or the TNF-α values were under the detection threshold. Striking differences were detected between treated and untreated cultures: the TNF-α value for non-treated cultures was 1350.2 pg/ml (expressed as mean of 3 determinations for both times, 18 and 24 hours) while TNF-α values for Lachesis-treated cultures was 290.0 pg/ml.

### Conclusion

Lachesis treatment of murine macrophages maintained in culture produced an activation of cell types that had no correspondence with an increase of TNF-α production. On the contrary, TNF-α values were significantly lower than those obtained from controlled cells, thus indicating that Lachesis was acting as a TNF-α synthesis inhibitor.

### Example 6

### Effects of a Highly Diluted Solution of Lachesis Venom in Patients with Rheumatoid Arthritis

Four patients suffering from rheumatoid arthritis received 5 ml of a highly diluted solution of Lachesis venom (10⁻⁷) PBS twice a day by inhalation route using an ultrasonic nebulizer device and 20 drops of the same solution administered sublingually (also twice a day) for 30 consecutive days. Clinical evaluations of pain, functionality and stiffness sub-scores of the WOMAC score (Western Ontario and McMaster Universities Osteoarthritis Index) were recorded weekly.

### Results

The main end-point, pain, was reduced in the 4 patients by day 7 after the start of therapy, showing a mean of 53.5 points +/−13.7 on the WOMAC score before Lachesis administration, and of 19.3+/−12.2 after Lachesis therapy. Functionality and stiffness sub-scores remained in low levels in the 4 patients studied. By the end of the study, quality of life was improved.

### Conclusions

Highly diluted Lachesis venom significantly ameliorates pain and stiffness while increasing joint functionality, thus rendering a better overall quality of life for these patients. Based on previous observations, one can speculate that Lachesis acts by lowering endogenous TNF-α levels.

### Example 7

### Effects of Lachesis Venom in Advanced Cancer Patients

In order to evaluate the effects of a highly diluted solution of Lachesis venom on terminally ill patients who are neoplastic, the following schedule was carried out. Seven male and seven post-menopausal women of differing ages were recruited, ranging from 48 to 76 years of age with a mean of 66 years. All were in an advanced stage of cancer beyond any therapeutic chance of improvement. The patients were not receiving any specific anti-neoplastic treatment at the moment of their recruitment.

All patients were informed of the scope of the present study and a written consent was obtained. On day 1, each patient was treated with 2 ml of PBS containing 30 pg/kg of Lachesis venom by inhalation using an ultrasonic nebulizer. The same schedule was repeated on day 5 and on day 10. Simultaneously, each patient received 30 drops of the same Lachesis solution sublingually, three times a day, for 14 consecutive days. Before the beginning of the study and then every 48 hours, each patient completed a specially designed questionnaire (see table 1). At the same time, a physical examination was performed, and weight, cardiac frequency, respiratory rhythm and blood pressure were recorded. At day 1, 7 and 14, plasma and urine biochemical analyses were performed (blood cell counts, hemoglobin percentage, eritrosedimentation rate, glycemia, urea and creatinine, transaminases, etc).

### Results

Of fourteen volunteers recruited, all of them finished the study. Before the beginning of the treatment, all men and five women expressed being tired; five men and six women presented feeling general pain; two men and a woman complained about nausea; five men and five women expressed signs of mild depression, five women and six men had a diminished capability to do anything that required minimal exertion, seven men and six women expressed a marked loss of appetite, with special aversion to meat, and three men and no woman showed signs of absenteeism. All volunteers expressed a marked loss of sexual desire.

No significant modifications could be recorded during and after treatment, although weight increased in all volunteers, reaching a mean of 2.1 kg, ranging from 200 grams to up to 6 kg. Only two men and a woman demonstrated better physical performance by the end of the trial, with increased endurance while performing job or domestic activities, but all volunteers except three men and three women expressed feeling less tired than at the beginning of the trial. 2 men and 2 women expressed no changes in their body pain, but the others expressed that they were feeling less pain. Two men and three women claimed to sleep better.

By the end of the treatment, most volunteers reported changes in their mood. They were more communicative, and wanted to chat with friends and relatives. They referred to being in “good humor,” and they paid more attention to TV and written news.

After treatment, three men and two women continued to be anorexic, while the remaining four men and five women showed hyperorexia. By day 8 after the beginning of treatment, six men and seven women expressed an important increase in the volume of food ingested. Most of them expressed a preference to eating meat and pasta. No volunteer expressed a minor sexual desire after treatment. Three men related a subjective increase in their libido with sexual fantasies.

Before the beginning of the treatment, all volunteers showed ferropenic anemia of different intensities. During treatment, a small improvement in this hematological parameter was recorded in three men and two women. By day 14, anemia improved in about 10% in five men and three women. Other blood counts showed no modifications. Glucose, urea and creatinine showed no modifications because of the treatment as well as CPK and LDH levels. TGP and TGO levels were high in three men and five women before starting the treatment. By day 14, TGP and TGO levels returned to normal values.

The table below contains questions about physical and emotional attitude that each participant was asked to complete. “BEFORE” is the term used to indicate records before starting treatment. “AFTER” is the term used to indicate records after treatment was started, not taking in to account the moment these signs were manifested during the treatment. Values are expressed as number of volunteers showing a given sign over the total number of volunteers of each gender.

| | MEN | | WOMEN | |
|---|---|---|---|---|
| QUESTIONS | Before | After | Before | After |
| Do you feel tired? | 7/7 | 2/7 | 5/7 | 3/7 |
| Do you feel any pain? | 5/7 | 2/7 | 6/7 | 2/7 |
| Do you have a loss of appetite? | 7/7 | 3/7 | 6/7 | 2/7 |
| Do you have an increase in your | | 4/7 | | 4/7 |
| appetite? | | | | |
| Do you prefer a special kind of food? | | 6/7 | | 6/7 |
| Do you eat more? | | 6/7 | | 7/7 |
| Did you have nausea? | 2/7 | 1/7 | 1/7 | 0/7 |
| Did you sleep better and more? | | 2/7 | | 3/7 |
| Did you feel in a bad mood? | 6/7 | 2/7 | 5/7 | 1/7 |
| Did you have difficulties interacting | 6/7 | 0/7 | 3/7 | 0/7 |
| w/people? | | | | |
| Were you in a good humor? | | 3/7 | | 2/7 |
| Were you not as nervous? | | 3/7 | | 5/7 |
| Did you want to chat with friends and | | 5/7 | | 6/7 |
| relatives? | | | | |
| Do you forget memories or lose things? | 2/7 | 0/7 | 0/7 | 0/7 |
| Did you feel lost? | 1/7 | 0/7 | 0/7 | 0/7 |
| Did you experience sexual fantasies? | 3/7 | | 0/7 | |
| Did you lose sexual desire? | 6/7 | 3/7 | 6/7 | 6/7 |

### Conclusions

Lachesis venom, administered by sublingual and inhalation routes and with the schedule just described, did not show any adverse effect on cancer patients. On physical examination, the most noteworthy fact was an increase in body weight, that was consistent with an increase in appetite and improvement in blood cell counts. Most volunteers related improvements in some parameters. These improvement were, in order of importance: increase in appetite, minor body pain, minor tiredness, better social relationship, better mood, increase in physical performance, and positive modifications on sexual desire.

It is known that many clinical manifestations of terminal cancer are due to an increase in circulating TNF-α, especially cachexia. Lachesis venom could revert some signs and symptoms of these cancer patients, possibly by means of a decay in TNF-α circulating levels.

### Example 8

### Effects of Lachesis Venom on AIDS Patients

TNF-α favors the transcription of messenger RNA of HIV by the production of nuclear factors that bind to HIV proteins. It is known that different concomitant infections in HIV+ patients produce an activation of macrophages, which in turn produce more TNF-α, rendering an increase in viral titers. In order to evaluate the effects of a highly diluted solution of Lachesis venom in the course of HIV infection in AIDS patients, the following trial was done.

### Material and Methods

Six male HIV+ patients aged more than 18 years or older with a CD4 count of 150 to 200 cells/ul, who were not on anti-retroviral therapy (ART) along with two male and two female HIV+ patients aged more than 18 years old with similar CD4 counts refractory to ART were recruited.

The volunteers were randomly divided into two groups: one group received Lachesis venom at a concentration of 30 pg/kg of body weight diluted in PBS by sublingual route 3 times a day for 180 days. The other group received Lachesis venom in a similar schedule plus ART. The patients were regularly followed at bi-monthly intervals with physical check-ups, assessment of any adverse effects, opportunistic infections and laboratory check-ups. CD4/CD8 counts were assessed at recruitment and then at bimonthly intervals using Becton Dickinson FACSCount system. HIV RNA levels (Viral Load) were assessed using Roche Amplicor HIV-1 Monitor Quantitative PCR Assay, Version 1.5 on Cobas Analyser, at recruitment and day 180. Results were compared with the patients' historical records.

### Results

All the volunteers had a feeling of general well-being throughout the whole study period. None of the patients developed any opportunistic infection or developed any demonstrable adverse event. All patents remained fully productive in their day-to-day life. All 10 volunteers gained weight. Weight gain ranged from 2 to 6 kg, while those patients receiving only Lachesis averaged weight gains of 3.11 kg. Those patients receiving Lachesis and ART had an average weight gain of 2.76 kg. In clinical practice, a body weight change of plus/minus 5% is considered a significant effect of treatment or illness. Both treatment groups are clinically significant, as they are >5% weight gain.

There was a significant difference in pre- and post-study CD4 lymphocyte counts in both sets of volunteers. There were no differences in CD4 counts between those patients receiving Lachesis alone and those receiving Lachesis plus ART. No increase in CD4 counts was noticed until day 60 post-treatment and counts were maintained at high levels until the end of the study. Comparing historical data, the increase in CD4 count was highly significant, since in patients not having ART therapy, lymphocytes showed a steep decline as time passed. Results are summarized in Tables 7 and 8. The viral load declined sharply in both groups of patients, but results were significant and surprising in those volunteers receiving Lachesis plus ART, since the decay was about 1 log of difference. These results are summarized in Table 9.

TABLE 7
CD4 count at different days post-treatment.
Values are expressed as cells/ul.

| Group | Day 0 | Day 60 | Day 120 | Day 180 |
|---|---|---|---|---|
| Lachesis | 170 | 304 | 302 | 298 |
| Lachesis + ART | 158 | 278 | 311 | 306 |

TABLE 8
CD4 count between Lachesis and Lachesis + ART
treated groups. Values are also compared with
historical data from previous studies.

| Group | Day 0 | Day 180 |
|---|---|---|
| Historical control | 183 | 161 |
| Lachesis | 170 | 298 |
| Lachesis + ART | 158 | 306 |

*Historical records were used because the design of the trial did not include a control group composed of patients receiving ART only (these patients were refractory to the drug).

TABLE 9
Viral load.

| Group | Day 0 | Day 180 |
|---|---|---|
| Lachesis | 126.392 | 65.094 |
| Lachesis + ART | 43.983 | 5.789 |

There are several conclusions from the results described above. Subjects receiving Lachesis venom treatment resulted in an increase in CD4 count and in body weight. Lachesis venom decreased HIV load in a significant manner in ART-naïve patients and in a highly significant way in ART-resistant patients. Lachesis venom treatment showed an improvement in generalized well-being and in quality of life among HIV/AIDS patients with CD4 lymphocyte counts between 150 and 200 cells/ul. Lachesis venom treatment improved the immune status of these patients. Lachesis was found effective in preventing the onset of opportunistic infections in HIV/AIDS patients. Lachesis venom treatment was shown to be an effective adjuvant for ART.

### OTHER EMBODIMENTS

Any improvement may be made in part or all of the compositions and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method for modulating circulating TNF-α levels in an animal subject, the method comprising the step of administering a composition comprising Lachesis venom or at least one component thereof and at least one of a pharmaceutically acceptable carrier and a pharmaceutically acceptable excipient to an animal subject having a disease or condition in which circulating TNF-α levels are elevated, wherein the composition is administered to the animal subject in an amount sufficient to decrease circulating TNF-α levels in the animal subject.

2. The method of claim 1, wherein the animal subject has at least one condition selected from the group consisting of: cancer, Chagas' disease, rheumatoid arthritis, cisplatin nephrotoxicity, sepsis, and an immunodeficiency disease.

3. The method of claim 1, wherein the animal subject is a human, cat, dog or horse.

4. The method of claim 2, wherein the immunodeficiency disease is acquired immune deficiency syndrome.

5. The method of claim 2, wherein the composition is an adjuvant in combination therapy.

6. The method of claim 1, wherein the at least one component is isolated from Lachesis venom.

7. The method of claim 1, wherein the at least one component is produced by recombinant DNA technology.

8. The method of claim 1, wherein the Lachesis venom or at least one component thereof is at a concentration of about 30 to about 320 picogram/kilogram.

9. The method of claim 1, wherein the composition comprises Lachesis venom in an aqueous solution.

10. The method of claim 1, wherein the composition is administered intravenously.
